(19) United States
(12) Patent Application Publication
(10) Pub. No.: US 2018/0055752 A1
(43) Pub. Date: Mar. 1, 2018

(54) TOPICAL COMPOSITIONS TO REDUCE HAIR GROWTH

(71) Applicant: The Regents of the University of California, Oakland, CA (US)

(72) Inventors: Ron S. Newfield, San Diego, CA (US); Lynne M. Bird, Coronado, CA (US); Sheila F. Friedlander, Del Mar, CA (US); Jaime J. Barea, San Diego, CA (US)

(73) Assignee: The Regents of the University of California, Oakland, CA (US)

(21) Appl. No.: 15/685,828

(22) Filed: Aug. 24, 2017

Related U.S. Application Data
(60) Provisional application No. 62/379,853, filed on Aug. 26, 2016.

(51) Int. Cl. A61K 8/46; A61Q 7/02; A45D 26/00; A61B 18/20

(52) U.S. Cl. CPC A61K 8/46; A61Q 7/02; A45D 26/0004; A45D 26/0014; A61B 2018/00476; A61B 18/203; A61K 2800/83; A61K 2800/805; A45D 26/0066

(57) ABSTRACT

This document relates to materials and methods for topical administration of sulfonylureas to inhibit, reduce, or prevent hair growth the topical composition being formulated for application to the skin of a mammal. The materials and methods of the disclosure may be used to inhibit, reduce, or prevent any unwanted hair growth including normal growth, and excessive hair growth.

### CROSS-REFERENCE TO RELATED APPLICATIONS

This application claims the benefit of U.S. Provisional Application Ser. No. 62/379,853, filed Aug. 26, 2016. The disclosure of the prior application is considered part of (and is incorporated by reference in) the disclosure of this application.

### BACKGROUND

### 1. Technical Field

This document relates to materials and methods for topical administration of sulfonylureas to inhibit, reduce, or prevent hair growth, with the topical composition being formulated for application to the skin of a mammal. The materials and methods of the disclosure may be used to inhibit, reduce, or prevent unwanted hair growth, including normal hair growth and excessive hair growth.

### 2. Background Information

A large unmet need exists for safe, simple treatments of excessive hair disorders which can have severe psychological effects, impacting an individual's self-esteem and ability to interact successfully in both the workplace and social settings. Currently there is no effective medical drug therapy for hypertrichosis. Current therapies with a potential for long-term hair removal include laser therapy and/or electrolysis, which require multiple courses, are expensive and uncomfortable, and are not fully or permanently effective. Waxing or shaving is often painful, may cause skin infection, and only offers short term relief.

### SUMMARY

This document provides materials and methods for topical administration of a sulfonylurea to inhibit, reduce, or prevent hair growth. The materials and methods of the disclosure may be used to inhibit, reduce, or prevent any unwanted hair growth including normal hair growth, and excessive hair growth, both of which can cause significant distress, particularly when present on the face and/or trunk.

This document includes topical compositions formulated for application to the skin of a mammal that include a sulfonylurea and can be used to inhibit, reduce, or prevent hair growth. The topical compositions can be formulated for epicutaneous application.

The sulfonylurea can target a potassium gated (K-ATP) channel. In some cases, the K-ATP channel comprises a SUR1/Kir6.2 K-ATP channel. In some cases, the K-ATP channel comprises a SUR2B/Kir6.1 K-ATP channel. The sulfonylurea can be selected from the group consisting of carbutamide, acetohexamide, chlorpropamide, tolbutamide, glipizide, gliclazide, glibenclamide, glyburide, glibornuride, gliquidone, glisoxepide, glyclopyramide, and glimepiride. In some cases, the sulfonylurea is glyburide.

The topical compositions can be pharmaceutically acceptable compositions. The pharmaceutically acceptable topical compositions can include one or more of ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, alcohol-based substances, polyethylene glycol, sodium carboxymethylcellulose, polyacrylates, waxes, polyethylene-polyoxypropylene-block polymers, wool fat, petrolatum oil, and mineral oil.

This document also includes methods for using topical compositions formulated for application to the skin of a mammal that include a sulfonylurea and can be used to inhibit, reduce, or prevent hair growth. A method for reducing, preventing, or inhibiting hair growth can include contacting the skin of a mammal with a topical composition comprising a sulfonylurea. In some cases, contacting the skin can be an epicutaneous application of the compositions. In some cases, the mammal can be a human. In some cases, the sulfonylurea targets a potassium gated (K-ATP) channel (e.g., a SUR1/Kir6.2 K-ATP channel or a SUR2B/Kir6.1 K-ATP channel). In some cases, the sulfonylurea is glyburide.

In some embodiments, the methods for using topical compositions formulated for application to the skin of a mammal that include a sulfonylurea and can be used to inhibit, reduce, or prevent hair growth in the anagen phase.

In some embodiments, the methods for using topical compositions formulated for application to the skin of a mammal that include a sulfonylurea and can be used to inhibit, reduce, or prevent hair growth due to hypertrichosis. In some cases, the hypertrichosis can be due to hirsutism. In cases where the hypertrichosis is due to hirsutism, the method can also include administering to the mammal one or more hormonal therapies (e.g., oral contraceptive pills, glucocorticoids, or antiandrogens).

In some cases, the hypertrichosis can be chemically induced. Chemicals that can induce hypertrichosis include, for example, diazoxide, testosterone, danazol, anabolic steroids, glucocorticoids, cyclosporine, minoxidil, and phenytoin. In some cases, the chemical hypertrichosis can be caused by diazoxide.

In some cases, the hypertrichosis can be an idiopathic hypertrichosis. Diseases and/or disorders that can induce hypertrichosis include Cantú syndrome.

The methods for using topical compositions formulated for application to the skin of a mammal that include a sulfonylurea and can be used to inhibit, reduce, or prevent hair growth can also include removing hair from the contacted skin. In some cases, the hair is removed via electrolysis. In some cases, the hair is removed via one or more of shaving, a depilatory, and friction. In some cases, the hair is removed via an epilation technique (e.g., tweezing, waxing, sugaring, or threading). In some cases, the hair is removed via a photoepilation technique (e.g., laser hair removal, intense pulsed light, or diode epilation).

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DETAILED DESCRIPTION

The present disclosure is directed to materials and methods for topical administration of sulfonylureas to inhibit, reduce, or prevent hair growth. The materials and methods of the disclosure may be used to inhibit, reduce, or prevent any unwanted hair growth including normal hair growth, and excessive hair growth, both of which can cause significant distress, particularly when present on the face and/or trunk.

The present disclosure includes a topical composition for inhibiting, reducing, or preventing hair growth which includes a sulfonylurea. Applying a topical sulfonylurea is a simple process, which is inexpensive compared to other hair removal methods (e.g., laser therapy or electrolysis), which often need to be repeated. It may be also possible to combine topical sulfonylurea therapy with secondary hair removal methods to achieve an optimal and long lasting effect.

All sulfonylureas contain a central S-phenylsulfonylurea structure with a p-substituent on the phenyl ring (R) and various groups terminating the urea N′ end group (R2). The general formula of a sulfonylurea is shown below.

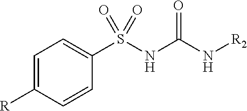

Sulfonylureas are known bind to an ATP-sensitive K⁺ (K-ATP) channel on the cell membrane of pancreatic beta cells. This inhibits a tonic, hyperpolarizing efflux of potassium, thus causing the electric potential over the membrane to become more positive. This depolarization opens voltage-gated Ca²⁺ channels. The K-ATP channel is an octameric complex consisting of an inner ring and outer ring. The inner ring includes four inward-rectifier potassium ion channel (Kir6) subunits and the outer ring includes four sulfonylurea receptor (SUR) subunits. The SUR subunits can affect channel activity in response to intracellular nucleotides or drugs. The K-ATP channels in the beta-cells in the pancreas are composed of SUR1 (ABCC8 gene) and Kir6.2 (KCNJ11 gene) and these channels control insulin release. Sulfonylurea drugs which block the K-ATP channel in the beta cells have been in clinical use for many decades to treat diabetes mellitus. Cantu syndrome is due to a mutation in the ABCC9 gene (van Bon et al., 2012 Am J Hum Genet. 90(6):1094-101; Harakalova et al., 2012 Nat Genet. 44(7):793-6) coding for SUR2. SUR2 forms a K-ATP channel together with Kir 6.1 (KCNJ8 gene), which has also been found to cause Cantu syndrome in a few cases when it was mutated, (Brownstein et al., 2013 Eur J Med Genet. 56(12):678-82). Alternative RNA splicing of the terminal exon of ABCC9 produces two SUR2 isoforms: SUR2A, predominantly expressed in cardiac and skeletal muscle cells, and SUR2B in smooth muscle. The existence of such isoforms explains the congenital cardiac anomalies seen in some patients with Cantu syndrome.

The sulfonylurea class of drugs, can also act on K-ATP channels present in the skin, which control hair growth at multiple sites in the cutaneous hair follicule. When human hair bulb tissues were isolated and evaluated, epithelial matrix expressed SUR1 and Kir6.2, whereas both dermal papilla and sheath exhibited SUR2B and Kir6.1 (Shorter et al., 2008 FASEB J. 22(6):1725-36). Experiments in deer hair (Davies et al., 2005 J Invest Dermatol. 124(4):686-94), showed that tolbutamide (1 mM), which selectively inhibits Kir6.2/SUR1 K-ATP channels with low potency, was able to inhibit hair follicle growth and abolished the effect of 10 μM minoxidil, and diazoxide. In contrast, glibenclamide (aka Glyburide) nonselectively blocks both SUR1 and SUR2B forms with high affinity, but did not inhibit hair follicle growth, but was able to counteract the effect of minoxidil.

In some aspects, a sulfonylurea binds to a K-ATP channel including SUR1/Kir6.2 subunits. In some aspects, a sulfonylurea binds to a K-ATP channel including SUR2B/Kir6.1 subunits.

As used herein, a sulfonylurea can be any molecule that binds to an K-ATP channel and includes, for example, carbutamide, acetohexamide, chlorpropamide, tolbutamide, glipizide (glucotrol), gliclazide, glibenclamide, glyburide (micronase), glibornuride, gliquidone, glisoxepide, glyclopyramide, glimepiride (amaryl), gliclazide MR (DIAMICRON MR60), and light sensitive sulfonylureas including JB253 and JB558. Light sensitive suflonylurueas are described in Broichhagen et al., 2014 Nature Communications 5:5116; and Broichhagen et al., 2015 Chemical Communications 51:6018. In some aspects the sulfonylurea is glyburide.

The composition described herein is a topical composition. A topical composition is a composition that is applied to body surfaces such as the skin or mucous membranes to treat ailments via a large range of classes including but not limited to creams, foams, gels, lotions, and ointments. Topical application of the composition eliminates or at least minimizes systemic side effects, primarily low blood glucose levels (hypoglycemia) typically associated with sulfonylurea treatment. A topical composition may be epicutaneous (applied directly to the skin), or applied to the surface of tissues other than the skin, such as eye drops applied to the conjunctiva, ear drops placed in the ear, or medications applied to the surface of a tooth. In some aspects the topical composition of the present disclosure is formulated for epicutaneous application. For example, epicutaneous composition can be applied using a patch (e.g., a transdermal patch).

The composition described herein can be applied therapeutically, i.e., after the hair growth has taken place. The composition described herein may also be applied prophylactically to avoid excess hair growth secondary to some medications (e.g. diazoxide and cyclosporine).

In some aspects the materials and methods of this disclosure inhibit, reduce, or prevent normal, but unwanted, hair growth. The hair cycle includes the anagen phase (hair growth), the catagen phase (hair follicle death), and the telogen phase (hair shedding). In embodiments where the materials and methods of this disclosure inhibit, reduce, or prevent normal, but unwanted, hair growth, the hair cycle is in the anagen phase and the anagen phase is inhibited, reduced, or prevented.

In some aspects the materials and methods of this disclosure inhibit, reduce, or prevent excessive hair growth. Excessive hair growth may be due to by any of a number of conditions. Conditions with excess hair growth include without limitation hypertrichosis (abnormal hair growth anywhere on the body), hirsutism (abnormal hair growth linked to excessive hormones), or a change in the hair cycle (the anagen phase increases beyond what is normal).

Hypertrichosis may be congenital (present at birth and typically caused by genetic mutations) or acquired later in life. Hypertrichosis may be hirsutism, a type of hypertrichosis characterized by male-pattern hair growth in women or children (congenital or acquired) caused by excess amounts of male hormones called androgens, primarily testosterone. Acquired hypertrichosis may be, for example, idiopathic, associated with a disease and/or disorder, chemical, or a combination thereof.

In some cases, hypertrichosis can be associated with a disease and/or disorder. For example, certain diseases and/or disorders, including without limitation, Cantú syndrome and Cushing syndrome, may cause excessive hair growth.

In some cases, hypertrichosis can be chemical. For example, the use of certain medicines, including without limitation diazoxide, testosterone, danazol, anabolic steroids, glucocorticoids, cyclosporine, minoxidil, and phenytoin may cause excessive hair growth as a side effect. For example, diazoxide can be used to lower blood pressure, by opening K-ATP channels in vascular smooth muscle SUR2B/Kir6.2, causing the vascular musculature to relax. It is also used to treat hyperinsulinism of infancy (Low et al., 1989 Aust Paediatr J. 25(3):174-7; Hussain et al., 2004 Pediatr Endocrinol Rev. 2 Suppl 1:163-7), which can result in lifethreatening hypoglycemia, by targeting the SUR1/Kir6.2 K-ATP channel in the beta-cells in the pancreas, and blocking insulin release. Unfortunately, it also acts on the K-ATP channels in the skin, thus causing severe hypertrichosis, which sometimes results in diazoxide discontinuation and need for another drug or often a near-total pancreatectomy. This invention would allow the systemic benefit of diazoxide to be realized (avoiding severe hypoglycemia) while avoiding the unwanted hypertrichosis.

In some cases, hypertrichosis can be a combination of causes. Certain diseases and/or disorders, for example, polycystic ovarian syndrome (PCOS), adrenal gland cancer, ovarian cancer, and congenital adrenal hyperplasia, hyperthecosis include endogenous hormonal effects which may cause excessive hair growth.

The composition includes an effective amount of a sulfonylurea. For example, an effective amount of a sulfonylurea can be about 1 mM. An effective amount of a topical composition including a sulfonylurea can be any amount that inhibits, reduces, or prevents hair growth (e.g., hypertrichosis) without producing significant toxicity to the mammal. The ability of the topical composition to inhibit, reduce, or prevent hair growth can be assessed by, for example, the character (hair length and thickness) of the hair. Toxicity to the mammal can be assessed by, for example, monitoring for systemic hypoglycemia. Sulfonylurea treatments are often associated with systemic side effects such as low blood glucose levels (hypoglycemia). One can easily monitor blood glucose levels (e.g., fasting and post-meal blood glucose levels) with a home glucometer, as used by patients with diabetes mellitus, and use lower concentration or volume of the drug to determine the effective amount. In addition, Cantu syndrome and diazoxide-induced hypertrichosis can serve as a models to determine the effective amount of a topical composition to block potassium channel-mediated hair growth. Effective amounts can vary depending on the severity of the hair growth, the route of administration, the age and general health condition of the subject, excipient usage, the possibility of co-usage with other therapeutic treatments, and the judgment of the treating physician.

In some cases, the composition including a sulfonylurea as described herein can be formulated into a pharmaceutically acceptable composition for administration to a mammal. For example, a therapeutically effective amount of a composition including a sulfonylurea can be formulated together with one or more pharmaceutically acceptable carriers, fillers, and/or vehicles. A pharmaceutical composition can be formulated for administration in solid or liquid form including, without limitation, sterile solutions, suspensions, sustained-release formulations, tablets, capsules, pills, powders, and granules. In some cases, a pharmaceutical composition (e.g., a sustained-release formulation) can be incorporated into a patch (e.g., a transdermal patch).

Pharmaceutically acceptable carriers, fillers, and vehicles that may be used in a pharmaceutical composition described herein include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, alcohol-based substances, polyethylene glycol, sodium carboxymethylcellulose, polyacrylates, waxes, polyethylene-polyoxypropylene-block polymers, wool fat, petrolatum oil, and mineral oil.

The present disclosure also includes methods for inhibiting, reducing, or preventing hair growth. Specifically, the methods for inhibiting or reducing hair growth as disclosed herein include contacting the skin of a mammal with a topical composition including a sulfonylurea. The sulfonylurea can be glyburide. The methods described herein function by targeting (i.e., interfering with specific molecules) a potassium gated (K-ATP) channel. In some cases, targeting can include binding to the target. The binding can be a noncovalent bond, a reversible covalent bond, or an irreversible covalent bond. The binding may induce a conformation change in the target. The binding may prevent other molecules from binding the target. In some cases, the K-ATP channel includes SUR1/Kir6.2 subunits. In some cases, the K-ATP channel includes SUR2B/Kir6.1 subunits. The contacting can be topical application. In some cases, the contacting is an epicutaneous application.

Any appropriate mammal can be treated as described herein. For example, humans, non-human primates, monkeys, horses, bovine species, porcine species, dogs, cats, mice, and rats having cancer can be treated to inhibit, reduce, or prevent hair growth. In some aspects, the mammal is a human.

In some embodiments, the methods can be used to inhibit, reduce, or prevent hair growth in the anagen phase.

In other embodiments, the methods can be used to inhibit, reduce, or prevent excessive hair growth. The treatment of, in particular long-term inhibition, reduction, or prevention, of excessive hair growth (e.g., hypertrichosis or hirsutism) has a large market.

The methods can be used to inhibit, reduce, or prevent hypertrichosis. As discussed herein, hypertrichosis may be congenital or acquired (idiopathic, chemical, or a combination thereof) later in life. The method can be used to inhibit, reduce, or prevent acquired hypertrichosis. In some instances, the method can be used to inhibit, reduce, or prevent excessive hair growth associated with chemically induced (e.g., by diazoxide) hypertrichosis. In some instances, method can be used to inhibit, reduce, or prevent excessive hair growth associated with idiopathic hypertrichosis. In some instances, method can be used to inhibit, reduce, or prevent excessive hair growth associated with hypertrichosis associated with Cantú syndrome. The methods of this disclosure may also include removing hair from the contacted skin by one or more hair removal methods.

The methods can be used to inhibit, reduce, or prevent hirustism. Hirsutism may respond to hormonal therapies, such as oral contraceptive pills (OCPs), glucocorticoids, or antiandrogens such as spironolactone, but may not result in full resolution. In cases where the hypertrichosis is hirsutism, the method can also include administration of one or more hormonal therapies. Examples of hormonal therapies include OCPs, glucocorticoids, or antiandrogens with medications that reduce androgen levels, such as antiandrogens. Antiandrogens alter the androgen pathway by blocking the appropriate receptors, competing for binding sites on the cell's surface, or affecting androgen production. Exemplary antiandrogenic drugs include spironolactone and cyproterone acetate. Some women may not tolerate the medications (e.g. OCPs) due to side effects.

In some cases, the methods can also include removing hair from the contacted skin by one or more hair removal methods. Hair removal methods are well known and can include electrolysis, depilation techniques, epilation techniques, or photoepilation techniques.

In some aspects, the methods for inhibiting, reducing, or preventing hair growth, as disclosed herein, can also include removing hair from the contacted skin by electroylsis.

Depilation techniques (removal of the part of the hair above the surface of the skin) include both mechanical methods such as, without limitation, shaving, friction, or trimming, and chemical methods such as, without limitation, compounds (creams or powders) which chemically dissolve hair. In some aspects, the methods for inhibiting, reducing, or preventing hair growth, as disclosed herein, can also include removing hair from the contacted skin by a depilation technique.

Epilation techniques (removal of the entire hair, including the part below the skin) include, for example, tweezing, waxing, sugaring, and threading. In some aspects, the methods for inhibiting, reducing, or preventing hair growth, as disclosed herein removing hair from the contacted skin by an epilation technique.

Photoepilation techniques include, for example, laser hair removal, intense pulsed light, and diode epilation. In some aspects, the methods for inhibiting, reducing, or preventing hair growth, as disclosed herein, can also include removing hair from the contacted skin by a photoepilation technique.

The invention will be further described in the following examples, which do not limit the scope of the invention described in the claims.

### EXAMPLES

### Example 1

### Topical Sulfonylurea as a Novel Therapy for Hypertrichosis Secondary to Diazoxide, and Potentially for Other Conditions with Excess Hair Growth

### Abstract

It is hypothesized that a topical application of a sulfonylurea drug, which can inhibit the ATP-sensitive potassium-gated channels (Kir6.X/SUR) present in human hair bulb tissues, will inhibit hair growth in a targeted manner. Diazoxide is used to treat severe hypoglycemia due to hyperinsulinism of infancy. However, this often results in hypertrichosis that can be severe enough to prevent its use. Diazoxide blocks insulin release from the pancreas by opening the SUR1/Kir6.2 channels in beta cells. Diazoxide can also act on two potassium-gated channels in the skin that affect hair growth, namely SUR1/Kir6.2, and SUR2B/Kir6.1, thus causing hypertrichosis. It is proposed that a topical sulfonylurea will inhibit the excessive hair growth due to diazoxide, but will not impact the beneficial effects of diazoxide on beta cells. This approach can also be applied to rare cases of Cantu syndrome, caused by mutations in ABCC9 (coding for SUR2) or in KCNJ8 (coding for Kir6.1) that is characterized by congenital hypertrichosis. More importantly, this approach may also be effective in treating other forms of hypertrichosis or hirsutism, that are quite common, yet very distressing to patients worldwide.

### BACKGROUND

Soon after diazoxide was first used as a hypertensive agent in the 1960s, reports about associated hypertrichosis surfaced (Okun et al., 1963 Arch Intern Med. 112:882-8). When diazoxide was used for the treatment of hypoglycemic states, hypertrichosis was noted as a frequent side effect. A case series of eight children treated with diazoxide for hypoglycemia of infancy, reported that all developed hypertrichosis (Koblenzer et al., 1968 Ann N Y Acad Sci. 150:373-82). Diazoxide is still the first line of treatment in hyperinsulinemia of infancy (HI) but its use is often limited by hypertrichosis, which can be severe and diffuse (Mohamed et al., 2012 J Clin Res Pediatr Endocrinol. 4:169-81; Hussain et al., 2004 Pediatr Endocrinol Rev. 2(Suppl 1):163-7; and Yorifuji et al., 2014 Ann Pediatr Endocrinol Metab. 19:57-68). The mechanism of action of diazoxide in HI has been well delineated. The K_{ATP} channels in the B-cells in the pancreas are composed of SUR1 (ABCC8 gene) and Kir6.2 (KCNJ11 gene) and these channels control insulin release in a glucose-dependent manner (Yorifuji et al., 2014 Ann Pediatr Endocrinol Metab. 19:57-68; and Snider et al., 2013 J Clin Endocrinol Metab. 98:E355-63). In HI this glucose-dependent regulation is absent, causing life-threatening hypoglycemia (Mohamed et al., 2012 J Clin Res Pediatr Endocrinol. 4:169-81; Yorifuji et al., 2014 Ann Pediatr Endocrinol Metab. 19:57-68; and Snider et al., 2013 J Clin Endocrinol Metab 98:E355-63). Diazoxide acts at the SUR1 and causes the SUR1/Kir6.2 K_{ATP} channel in the β-cells to open, thus blocking insulin release (Yorifuji et al., 2014 Ann Pediatr Endocrinol Metab. 19:57-68). Diazoxide-unresponsiveness depends on the severity of the mutation affecting the K_{ATP} channel (Yorifuji et al., 2014 Ann Pediatr Endocrinol Metab. 19:57-68; and Snider et al., 2013 J Clin Endocrinol Metab. 98:E355-63). In contrast, Sulfonylurea drugs, which block the K_{ATP} channel in the B-cells, have been in clinical use for many decades to treat type 2 diabetes mellitus (Melander et al., 1990 Diabetes Care. 13(Suppl 3):18-25), and more recently have been used successfully in neonatal diabetes as well (Babenko et al., 2006 N Engl J Med. 355:456-66; and Rafiq et al., 2008 Diabetes Care. 31:204-9), by enhancing insulin release. K_{ATP} channels consist of two rings, each consisting of 4 subunits. The inner ring of four inwardly rectifying K⁺ channel (Kir6.X) subunits forms the pore through which potassium ions pass. The outer ring consists of four regulatory sulfonylurea receptor (SUR) subunits that can affect channel activity in response to intracellular nucleotides or drugs (Shorter et al., 2008 FASEB J. 22:1725-36; Bryan et al., 2007 Pflugers Arch. 453:703-18; and Flagg et al., 2010 Physiol Rev. 90:799-829). Both subunit types are required to form a functional channel. There are 2 SUR subunits, SUR1 and SUR2, and the latter has 2 splice variants SUR2A and SUR2B. SUR2A is predominantly expressed in cardiac and skeletal muscle cells, and SUR2B in smooth muscle (Bryan et al., 2007 Pflugers Arch. 453:703-18; and Flagg et al., 2010 Physiol Rev. 90:799-829). These different tissues have different combinations of the 2 types of subunits. Of note, the genes for Kir6.1 and SUR2 are located in a cluster of genes (12p12.1), suggesting co-regulation at the gene level (Flagg et al., 2010 Physiol Rev. 90:799-829). Similarly, the genes for Kir6.2 and SUR1 are also located in a cluster of genes but on chromosome 11p15.1, and mutations in those 2 genes lead to HI (Snider et al., 2013 J Clin Endocrinol Metab. 98:E355-63) and neonatal diabetes (Babenko et al., 2006 N Engl J Med. 355:456-66; and Rafiq et al., 2008 Diabetes Care. 31:204-9). Although the presence of K_{ATP} channels in hair follicles has been previously described and the different subunit compositions were characterized (Shorter et al., 2008 FASEB J. 22:1725-36; and Davies et al., 2005 J Invest Dermatol. 124:686-94), the significance of this finding was not well appreciated. Cantú syndrome, or hypertrichotic osteochondrodysplasia (MIM 239850) is a rare autosomal dominant condition characterized by congenital hypertrichosis, distinctive facial features and cardiac defects, as well as other anomalies. It was recently discovered to be due to SUR2 (ABCC9 gene) mutations (Harakalova et al., Nat Genet. 44:793-6; and van Bon et al., Am J Hum Genet. 90(6):1094-101), or less often due to Kir 6.1 (KCNJ8 gene) mutations (Brownstein et al., 2013 Eur J Med Genet. 56:678-82). The similarities between the phenotype of the rare individuals with Cantú syndrome and the excess hair growth from the K_{ATP} channel agonist minoxidil indicate that the mutations in ABCC9 result in channel opening (Harakalova et al., Nat Genet. 44:793-6; and van Bon et al., Am J Hum Genet. 90(6):1094-101), which was supported by electrophysiological measurements (Harakalova et al., Nat Genet. 44:793-6). This is yet another rare condition that expands our understanding, and in this case, the connection between hypertrichosis and abnormalities in, or drugs affecting potassium-gated K_{ATP} channels. This opens up the possibility of using sulfonylurea drugs to treat hypertrichosis due to diazoxide, in the rare cases of Cantú syndrome, or in more common conditions characterized by excess hair.

### The Hypothesis

The hypothesis states that a topical sulfonylurea can inhibit the K_{ATP} potassium-gated channels present in skin that control human hair growth, and thus reduce excessive hair growth in a localized, specific and targeted manner. A topical sulfonylurea can help control hypertrichosis that is due to either a mutation (Cantú syndrome) or a drug (e.g. diazoxide) affecting the activity of the K_{ATP} channels (Kir6.X/SUR) present in the hair bulbs that control hair growth. Treatment of hypertrichosis and hirsutism due to other, more common causes may be amenable to such a therapeutic approach. This will present a real breakthrough, as many such patients lack effective medical intervention, and resort to costly interventions, such as laser treatments, or electrolysis.

### Evaluation of the Hypothesis/Idea

Since hypertrichosis and cardiac manifestations are key features of Cantú syndrome, and in light of the similarities between this rare syndrome and the actions of minoxidil, it focused the attention of the biomedical community on the potential use of a sulfonylurea, a K_{ATP} channel antagonist, as a potential drug for Cantú syndrome (Harakalova et al., Nat Genet. 44:793-6; and van Bon et al., Am J Hum Genet. 90(6):1094-101). Knowledge about the tissue distribution of K_{ATP} channels with different subunit compositions (Shorter et al., 2008 FASEB J. 22:1725-36; and Davies et al., 2005 J Invest Dermatol. 124:686-94) preceded the identification of the mutations in the ABCC9 gene being the cause of Cantú syndrome. When human hair bulb tissues were isolated and evaluated, epithelial matrix expressed SUR1 and Kir6.2, whereas both dermal papilla and sheath exhibited SUR2B and Kir6.1 (Shorter et al., 2008 FASEB J. 22:1725-36). The distribution of these K_{ATP} channels in human hair, and the molecular defects noted in Cantú syndrome explain the severe hypertrichosis seen in Cantú syndrome. Earlier experiments in deer hair (Davies et al., 2005 J Invest Dermatol. 124:686-94), showed that the sulfonyurea drug Tolbutamide (1 mM), which selectively inhibits Kir6.2/SUR1 K_{ATP} channels with low potency, was able to inhibit hair follicle growth and abolished the effect of 10 μM minoxidil, and diazoxide. In contrast, glibenclamide (a different sulfonylurea, also known as Glyburide) non-selectively blocks both SUR1 and SUR2B forms with high affinity, was tested at a single concentration, but did not inhibit deer hair follicle growth, yet was able to counteract the effect of minoxidil. Similar experiments in human hair follicles were performed only with tolbutamide and minoxidil, but not with glibenclamide. Tolbutamide was shown to shorten anagen in cultured human hair follicles, an effect that was blocked by minoxidil (Shorter et al., 2008 FASEB J. 22:1725-36). This prompted the authors to speculate on a potential role for K_{ATP} channel antagonists to treat hirsutism (Shorter et al., 2008 FASEB J. 22:1725-36). This lends further support to this hypothesis, although the authors did not specifically address the possible role a sulfonylurea might have in Cantú syndrome or in treating hypertrichosis secondary to diazoxide. Neonatal diabetes due to mutations in the ABCC8 gene coding for SUR1 can be treated successfully with sulfonylurea drugs. Some of the ABCC9 mutations noted in Cantú syndrome are homologous to the ABCC8 mutations noted in neonatal diabetes cases responding to sulfonylurea (Babenko et al., 2006 N Engl J Med. 355:456-66; and Rafiq et al., 2008 Diabetes Care. 31:204-9), giving further evidence in support of this hypothesis.

Based on the type of K_{ATP} channels noted in human hair bulbs present in skin, we predict that topical application of a sulfonylurea drug that acts on both SUR1/Kir6.2 and SUR2B/Kir6.1 channels will be the optimal sulfonylurea to reduce hair growth (anagen phase). We plan to test the effect of two concentrations of a topical formulation of glyburide compounded for our proposed study with a vehicle that will aid in absorption, in two stages. Our design will include application of the drug with vehicle on one side of the back, and applying the vehicle alone at the contra-lateral area of the back, at the same level. The first stage, awaiting IRB approval, is a proof of concept pilot study, investigating the drug in a family with Cantú syndrome that is followed at our medical center, and in infants and young children with hyperinsulinism of infancy who are receiving diazoxide. We will include those treated with diazoxide long term that have developed moderate or severe hypertrichosis, and those that have just started on diazoxide and seem to respond to it in terms of control of their hypoglycemia. The fact that the patients with HI respond to diazoxide indicates that their K_{ATP} channels in the skin should respond to the study drug as well. This invention would allow the systemic benefit of diazoxide to be realized (avoiding severe hypoglycemia) while avoiding the unwanted hypertrichosis. Using home glucose meters we will closely monitor blood glucose levels of patients. This is to verify there are no systemic effects due to the topical application of glyburide. If this proof of concept stage would seem efficacious and safe, we will move to test the effect of this formulation in patients with other forms of hypertrichosis (e.g. ethnic, secondary to other medications) or hirsutism (e.g. androgen excess states).

### Consequences of the Hypothesis and Discussion

The importance of the hypothesis is that it views K_{ATP} as a therapeutic target for excess hair, whether due to a genetic channelopathy, exogenous drugs, or other conditions. The hypothesis broadens the spectrum of conditions amenable to intervention by targeting the K_{ATP} channels present in human skin, by applying it locally. In the case of diazoxide, which is administered to treat HI, this hypothesis proposes to use a topical sulfonylurea drug to avoid the cutaneous side effects of diazoxide and enable its use. This is similar to adding a potassium-sparing diuretic to avoid hypokalemia when using furosemide or a thiazide diuretic. If this hypothesis is confirmed in patients with Cantú syndrome and/or those receiving diazoxide, then a topical application of a sulfonylurea drug (e.g. glyburide) may be tried as a simple, affordable and painless alternative to laser, waxing or electrolysis, now used to treat hypertrichosis or hirsutism. Needless to say, this would be a significant advance in the treatment and possibly prevention of excess hair, which adversely impacts the self esteem and well being of many people across the world.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A topical composition comprising a sulfonylurea, wherein the topical composition inhibits, reduces, or prevents hair growth and wherein the topical composition is formulated for application to the skin of a mammal.

2. The topical composition of claim 1, wherein the sulfonylurea targets a potassium gated (K-ATP) channel.

3. The topical composition of claim 2, wherein the K-ATP channel comprises a SUR1/Kir6.2 K-ATP channel.

4. The topical composition of claim 2, wherein the K-ATP channel comprises a SUR2B/Kir6.1 K-ATP channel.

5. The topical composition of claim 1, wherein the sulfonylurea is selected from the group consisting of carbutamide, acetohexamide, chlorpropamide, tolbutamide, glipizide, gliclazide, glibenclamide, glyburide, glibornuride, gliquidone, glisoxepide, glyclopyramide, and glimepiride.

6. The topical composition of claim 1, wherein the sulfonylurea is glyburide.

7. The topical composition of claim 1, wherein the topical composition is formulated for epicutaneous application.

8. The topical composition of claim 8, wherein the topical composition is pharmaceutically acceptable.

9. The topical composition of claim 8, wherein topical composition comprises one or more of ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, alcohol-based substances, polyethylene glycol, sodium carboxymethylcellulose, polyacrylates, waxes, polyethylene-polyoxypropylene-block polymers, wool fat, petrolatum oil, and mineral oil.

10. A method for reducing, preventing, or inhibiting hair growth, the method comprising:
contacting the skin of a mammal with a topical composition comprising a sulfonylurea.

11. The method of claim 10, wherein the contacting the skin is epicutaneous application.

12. The method of claim 11, wherein said mammal is a human.

13. The method of claim 11, wherein the hair growth is in the anagen phase.

14. The method of claim 11, wherein the hair growth is due to hypertrichosis.

15. The method of claim 14, wherein the hypertrichosis is due to hirsutism.

16. The method of claim 15, further comprising administering to the mammal one or more hormonal therapies selected from the groups consisting of oral contraceptive pills, glucocorticoids, or antiandrogens.

17. The method of claim 11, wherein the hypertrichosis is chemically induced.

18. The method of claim 17, wherein the chemical is selected from the group consisting of diazoxide, testosterone, danazol, anabolic steroids, glucocorticoids, cyclosporine, minoxidil, and phenytoin.

19. The method of claim 11, wherein the hypertrichosis is an idiopathic hypertrichosis.

20. The method of claim 19, wherein the idiopathic hypertrichosis is Cantú syndrome.

21. The method of claim 11, wherein the hypertrichosis is a chemical hypertrichosis.

22. The method of claim 11, comprising removing hair from the contacted skin.

23. The method of claim 22, wherein the hair is removed via electrolysis.

24. The method of claim 22, wherein the hair is removed via one or more of shaving, a depilatory, and friction.

25. The method of claim 22, wherein the hair is removed via an epilation technique selected from the group consisting of tweezing, waxing, sugaring, and threading.

26. The method of claim 22, wherein the hair is removed via a photoepilation technique selected from the group consisting of laser hair removal, intense pulsed light, and diode epilation.

27. The method of claim 11, wherein the sulfonylurea targets a potassium gated (K-ATP) channel.

28. The method of claim 26, wherein the K-ATP channel comprises a SUR1/Kir6.2 K-ATP channel or a SUR2B/Kir6.1 K-ATP channel.

29. The method of claim 11, wherein the sulfonylurea is glyburide.

30. The method of claim 11, wherein the topical application is an epicutaneous application.
